(19) United States
(12) Patent Application Publication
(10) Pub. No.: US 2025/0352152 A1
(43) Pub. Date: Nov. 20, 2025

(54) MONITORING DEVICE, AND MONITORING INFORMATION DISPLAY METHOD AND APPARATUS

(71) Applicants: Lin TAN, Shenzhen (CN); Weiwei YUAN, Shenzhen (CN); Xiaoling ZOU, Shenzhen (CN)

(72) Inventors: Lin TAN, Shenzhen (CN); Weiwei YUAN, Shenzhen (CN); Xiaoling ZOU, Shenzhen (CN)

(21) Appl. No.: 19/279,981

(22) Filed: Jul. 24, 2025

Related U.S. Application Data
(63) Continuation of application No. 17/187,707, filed on Feb. 26, 2021.
(63) Continuation-in-part of application No. PCT/CN2018/102779, filed on Aug. 28, 2018.
Application No. 17/187,707 is a continuation-in-part of application No. PCT/CN2019/084208, filed on Apr. 25, 2019.

(30) Foreign Application Priority Data
Aug. 28, 2018 (CN) 201810990861.7

(51) Int. Cl. A61B 5/00; A61B 5/145

(52) U.S. Cl. CPC A61B 5/742; A61B 5/14542

(57) ABSTRACT

A method for displaying monitoring information includes acquiring measurement data of at least one physiological parameter of a monitored object, obtaining data of at least one physiological parameter within a pre-set time duration, performing distribution statistics on the data within the pre-set time duration based on at least one parameter-value partition, determining a distribution statistic result corresponding to the parameter-value partition where the parameter-value partition represents a numerical interval of the at least one physiological parameter, providing a monitoring interface, generating a measurement data display region and a distribution statistics display region at the monitoring interface, displaying the measurement data of at least one physiological parameter in the measurement data display region of the monitoring interface, and displaying the distribution statistic result in the distribution statistics display region of the monitoring interface.

### CROSS-REFERENCE TO RELATED APPLICATIONS

This application is a continuation of U.S. application Ser. No. 17/187,707, filed Feb. 26, 2021, which is a continuation-in-part of Patent Cooperation Treaty Application No. PCT/CN2018/102779, filed on Aug. 28, 2018, and Patent Cooperation Treaty Application No. PCT/CN2019/084208, filed on Apr. 25, 2019, which claims priority and benefits of Chinese Patent Application No. 201810990861.7, filed on Aug. 28, 2018. These applications are hereby incorporated by reference.

### TECHNICAL FIELD

This disclosure relates to the technical field of monitoring devices, and more particularly to a monitoring device, and a monitoring information display method and apparatus.

### BACKGROUND

In the medical field, it is necessary to monitor physical conditions of a patient. Specifically, a monitoring device can monitor the patient's physiological parameters through various measurement modules. These physiological parameters need to be provided to medical personnel to enable the medical personnel to determine whether the patient is in a normal state or an abnormal state according to the parameter values. If an abnormal state is observed, the medical personnel need to take corresponding medical aid measures in time to restore the physical state to normal.

Therefore, a technical solution is needed to display the monitored physiological parameters as a basis for the medical personnel to determine the patient's physical state.

### SUMMARY

In one aspect, a method for displaying monitoring information may be provided in this disclosure. Measurement data of at least one physiological parameter of a monitored object may be acquired by at least one sensor, and data of the at least one physiological parameter within a pre-set time duration may be obtained by a processor from the measurement data of the at least one physiological parameter. Distribution statistics may be performed on the data within the pre-set time duration according to at least one parameter-value partition, and a distribution statistic result can be determined corresponding to the parameter-value partition, where the parameter-value partition represents one or more numerical intervals of the physiological parameter. A monitoring interface can be provided, at which a measurement data display region and a distribution statistics display region are generated. The measurement data of the at least one physiological parameter can be displayed in the measurement data display region of the monitoring interface, and the distribution statistic result can be displayed in the distribution statistics display region of the monitoring interface.

In some embodiments, a distribution statistic chart/table may further be generated according to the parameter-value partition and the distribution statistic result, and the distribution statistic chart/table can be displayed in the distribution statistics display region.

In some embodiments, the distribution statistic chart/table can be a histogram with the parameter-value partition and the distribution statistic result as two coordinate axes; or, the distribution statistic chart/table can be a statistic table with the parameter-value partition as a header and with the distribution statistic result as a table content.

In some embodiments, the data within the pre-set time duration may include the data obtained by means of continuous measurement or discontinuous measurement between any two time points.

In some embodiments, the distribution statistic result may be a number or a ratio of two numbers of the data of the at least one physiological parameter within the pre-set time duration respectively falling into each numerical interval corresponding to the parameter-value partition.

In some embodiments, the parameter-value partition can include a treatment target partition which corresponds to a numerical interval of a treatment target value of the physiological parameter. The method may further include displaying a non-treatment-target partition with a first display pattern, and displaying the treatment target partition with a second display pattern that is different from the first display pattern.

In some embodiments, one target condition may be further determined from the at least one parameter-value partition according to setup information; a display pattern for a statistic result corresponding to the target condition can be determined to be different from that for other statistic results; and the distribution statistic result can be displayed in the distribution statistics display region of the monitoring interface based on the display pattern.

In some embodiments, a corresponding waveform chart and/or trend chart may further be generated according to the data of the at least one physiological parameter within the pre-set time duration, and the waveform chart and/or the trend chart may be displayed in the measurement data display region. Alternatively, a trend display region may be generated at the monitoring interface, and the trend chart and/or the trend table of the at least one physiological parameter can be displayed in the trend display region.

In some embodiments, the method may further include switching between the trend chart and the trend table displayed in the trend display region in response to an instruction input by a user.

In some embodiments, the trend chart and/or the trend table may be refreshed when displayed in the trend display region in response to a pre-set trigger condition. Alternatively, the distribution statistic result, the trend chart and/or the trend table may be synchronously refreshed after the measurement data of the physiological parameter is updated.

In some embodiments, parameter values of a physiological parameter in the trend table may be monitored, and the parameter value may be displayed based on a pre-set display pattern when the parameter value exceeds a corresponding parameter threshold. Alternatively, parameter values of a physiological parameter in the trend table may be monitored, and the parameter value may be displayed based on a pre-set display pattern when alarm information associated with the parameter value is monitored. Alternatively, the method may further include displaying the trend chart with a display pattern by which normal and abnormal parameter values of the physiological parameter in the trend chart can be differentiated.

In some embodiments, the measurement data may be displayed in the measurement data display region based on a pre-set display pattern when the measurement data exceeds a corresponding parameter threshold.

In some embodiments, a selection operation control may further be provided in the distribution statistics display region; and in response to a selection instruction input by a user through the selection operation control, the distribution statistic result of the physiological parameter to be displayed in the distribution statistics display region and a value of the pre-set time duration can be determined.

In some embodiments, the monitoring interface further includes a state indication region that displays at least one graphical state indicator with multiple indication blocks; each of the multiple indication blocks respectively corresponds to a parameter value ranges of the physiological parameter, and the multiple indication blocks are displayed in the state indication region in an orderly arrangement according to a numerical value of the parameter value ranges of the physiological parameter corresponding to the indication blocks. The method may further include determining a parameter value range to which the measurement data of the physiological parameter belongs; and indicating an indication block of the graphical state indicator corresponding to the parameter value range to which the measurement data belongs.

In some embodiments, the multiple indication blocks may include a target indication block which represents an expected state of the monitored object with respect to the at least one physiological parameter, and the target indication block is displayed in a display and output mode different from other indication blocks when the parameter value range to which the measurement data belongs is a parameter value range corresponding to the target indication block.

In some embodiments, an indication icon is displayed at a position of the indication block corresponding to the parameter value range to which the measurement data belongs.

In some embodiments, the multiple indication blocks may include a treatment target indication block corresponding to a parameter value range of a treatment target value of the physiological parameter. The method may further include: when the measurement data of the physiological parameter is determined to change from a parameter value range to which a non-treatment-target value belongs to a parameter value range to which the treatment target value belongs, the treatment target indication block is displayed in a preset display pattern different from an original display pattern.

In some embodiments, a parameter pointer may be further displayed in the state indication region, with a first end of the parameter pointer indicating a position of the indication block corresponding to the parameter value range to which the measurement data belongs.

In some embodiments, the multiple indication blocks of the graphical state indicator are arranged in a circular arc shape, and a second end of the parameter pointer is displayed at a center position corresponding to the circular arc.

In some embodiments, a display brightness of the treatment target indication block may be increased from an initial brightness value to a preset brightness value; and/or a display color of the treatment target indication block may be switched from an initial color to a preset color.

In some embodiments, the multiple indication blocks of the graphical state indicator may be displayed in different display patterns.

In some embodiments, in the graphical state indicator, the display brightness of the indication block corresponding to the parameter value range to which the measured value belongs can be increased from the initial brightness value to the preset brightness value.

In some embodiments, the display color of the indication block corresponding to the parameter value range to which the measured value belongs can be switched from the initial color to the preset color.

In some embodiments, the graphical state indicator may be in an arc shape or a straight-bar shape formed by connecting multiple indication blocks.

In some embodiments, the multiple indication blocks of the graphical state indicator can be arranged in a circular arc shape in an orderly manner, and the other end of the parameter pointer may be displayed at the center position corresponding to the circular arc.

In still another aspect, a method for displaying monitoring information may be provided in this disclosure. The method may include: obtaining, by at least one sensor, data of at least one physiological parameter of a monitored object within a pre-set time duration;

performing, by a processor, distribution statistics on the data within the pre-set time duration based on at least one parameter-value partition, and determining a distribution statistic result corresponding to the parameter-value partition, wherein the parameter-value partition represents one or more numerical intervals of the physiological parameter, and the numerical interval is determined based on one or more of an alarm threshold, a baseline range, and a treatment target range corresponding to the physiological parameter; providing a monitoring interface, and generating a distribution statistics display region at the monitoring interface; and displaying the distribution statistic result in the distribution statistics display region of the monitoring interface.

### BRIEF DESCRIPTION OF THE DRAWINGS

In order to more clearly illustrate the technical solutions in the embodiments of this disclosure or in the prior art, a brief introduction to the drawings required for the description of the embodiments or the prior art will be provided below. Obviously, the drawings in the following description are only some of the embodiments of this disclosure, and those of ordinary skilled persons in the art would also have been able to obtain other drawings from these drawings without involving any inventive effort.

FIG. 1 is a schematic flow diagram of a method for displaying monitoring information;

FIGS. 2A-2G are various schematic diagrams of a monitoring interface;

FIG. 3 is a schematic structural diagram of a monitoring information display apparatus; and

FIG. 4 is a schematic hardware structural diagram of a monitor.

FIG. 5 is a schematic flowchart of a method for displaying a physiological sign parameter;

FIG. 6 is a schematic diagram of a monitoring interface for oxyhemoglobin saturation;

FIG. 7 is a schematic diagram of a monitoring interface for pulse rate;

FIG. 8 is a schematic diagram of a monitoring interface for perfusion index;

FIG. 9 is a schematic diagram of a monitoring interface for oxyhemoglobin saturation, pulse rate and perfusion index; and

FIGS. 10A-10F are multiple schematic diagrams of the monitoring interface for oxyhemoglobin saturation.

### DESCRIPTION OF THE EMBODIMENTS

The technical solutions of the embodiments of this disclosure will be described below clearly in conjunction with the accompanying drawings of the embodiments of this disclosure. Obviously, the embodiments described are merely some of, rather than all of, the embodiments of this disclosure. Based on the embodiments in this disclosure, all the other embodiments that would have been obtained by those of ordinary skill in the art without any inventive effort shall fall within the scope of protection of this disclosure.

In the medical field, it is often necessary to monitor physical conditions of a patient. Therefore, it is necessary to monitor the patient's physiological parameters and display the same to medical personnel. The medical personnel observe the patient's abnormal state and give medical aid measures to the patient according to the physiological parameters.

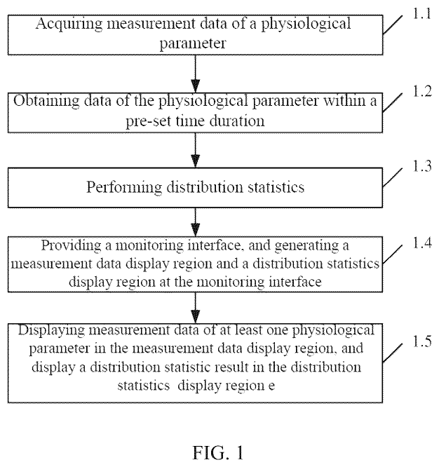

This disclosure provides a method for displaying monitoring information, which can be applied to various monitoring devices. As shown in FIG. 1, a process of the method for displaying the monitoring information specifically includes steps 1.1-1.5 as follows.

At step 1.1, measurement data of at least one physiological parameter is acquired from a monitored object. The measurement data of the physiological parameter may specifically be current measurement data.

The monitored object may be a patient object or an object with other monitoring needs, and the measurement data of the physiological parameter is acquired from the monitored object by means of at least one physiological sign sensor.

The measurement data of the physiological parameter of the monitored object can be collected using a sign parameter accessory device (such as a blood oxygen saturation probe), and can be stored in a memory. It should be noted that the measurement data of the physiological parameter may be either acquired from the memory, or be directly acquired from a collection module in this disclosure.

Therefore, the method provided in this disclosure can be applied not only to a bedside device, but also to a central station. When applied to the bedside device, the measurement data of the physiological parameter is acquired by means of accessory devices for various physiological parameters; and when applied to the central station, the central station acquires the measurement data of the physiological parameters from the bedside device through a network.

At step 1.2, data of the at least one physiological parameter within a pre-set time duration is obtained.

In addition to observing a real-time physiological state of the monitored object, the monitoring need further includes ascertaining a historical physiological state of the monitored object, which two should be combined to provide the medical personnel with more abundant diagnosis basis. Therefore, it is also necessary to obtain the data of the physiological parameter of the monitored object within the pre-set time duration.

It may be understood that there are a large amount of data of the physiological parameter within the pre-set time duration, but not all the data have guidance significance for the current diagnosis. Thus, according to the actual monitoring need, the time duration can be pre-set to limit the time within which the data need to be acquired. For ease of description, the set time duration can be referred to as the pre-set time duration.

It may be noted that the pre-set time duration is not limited in the form, which may be either a continuous historical time period or multiple discontinuous historical time points. That is, the data within the pre-set time duration may be the data obtained by continuous measurement between any two time points.

The pre-set time duration may either be pre-set by a monitoring system or be set by the user's own choice. Specifically, an input window can be provided at a monitoring interface for the user to input a setup instruction. For example, options of the pre-set time duration can be provided in the form of a drop-down list, and the user can select a certain option. In response to the setup instruction input by the user, the time duration set by the user is taken as the pre-set time duration.

At step 1.3, distribution statistics is performed on the data within the pre-set time duration based on at least one parameter-value partition, and a distribution statistic result is determined corresponding to the parameter-value partition, where the parameter-value partition represents one or more numerical intervals of the physiological parameter.

In order to facilitate the observation, the data within the pre-set time duration need to be analyzed and processed before being displayed. One way of analysis and processing is to classify the data within the pre-set time duration, that is, the parameter-value partition is pre-set, and the distribution statistics is performed on the data within the pre-set time duration according to the pre-set parameter-value partition. It may be noted that the parameter-value partition may be a segment of time, or a segment of the numerical value range of the data within the pre-set time duration, or a combination thereof, or other segment conditions.

When there are multiple parameter-value partitions, the respective distribution statistic result is obtained for each parameter-value partition after the distribution statistic. Of course, there may also be one parameter-value partition, and statistics are only performed on the data, falling into this parameter-value partition, among all the data within the pre-set time duration. For example, only a statistic histogram of a target parameter-value partition is displayed.

There may be many numerical values of the data within the pre-set time duration, and the numerical values may have different contents. The data within the pre-set time duration are displayed in segments, such that by means of comparing the distribution statistic results of different parameter-value partitions, the medical personnel can ascertain the change of the data within the pre-set time duration.

At step 1.4, a monitoring interface is provided, and a measurement data display region and a distribution statistics display region are generated at the monitoring interface.

At step 1.5, the measurement data of the at least one physiological parameter is displayed in the measurement data display region of the monitoring interface, and the distribution statistic result is displayed in the distribution statistics display region of the monitoring interface.

A displayer of a monitoring device provides the monitoring interface. The monitoring interface may be the entire monitoring interface of the displayer of the monitoring device, and may also be a window embedded in or suspended on the entire monitoring interface of the displayer.

The measurement data of the physiological parameter and the data within the pre-set time duration obtained as above are displayed in different regions of the monitoring interface, where the region where the measurement data is displayed is referred to as the measurement data display region, and the region where the distribution statistic result of the data within the pre-set time duration is displayed is referred to as the distribution statistics display region.

In an embodiment, the measurement data display region and the distribution statistics display region may have a binding relationship, and are displayed at the monitoring interface at the same time and disappear from the monitoring interface at the same time. The binding relationship can be reflected in the display position relationship, for example, the measurement data display region may be next to the distribution statistics display region in the monitoring interface.

It may be noted that there may be one or multiple physiological parameters. When there are multiple physiological parameters, the measurement data display region may be divided into multiple sub-regions that respectively display the measurement data of different types of physiological parameters, and similarly, the distribution statistics display region may also be divided into multiple sub-regions that respectively display the distribution statistic results of different types of physiological parameters.

In addition, if the measurement data of the physiological parameter and/or the data within the pre-set time duration exceed an alarm threshold corresponding to the physiological parameter, the distribution statistic result of the measurement data and/or the data within the pre-set time duration can be displayed in a display pattern corresponding to the alarm. That is, the measurement data of the physiological parameter are monitored, and if the measurement data exceeds the corresponding parameter threshold, the measurement data is displayed in the measurement data display region based on a pre-set alarm display pattern. The alarm display pattern may be any pre-set display pattern differentiated from a normal situation.

Moreover, the distribution statistic result displayed in the distribution statistics display region may be set by the user. Specifically, a selection operation control is provided in the distribution statistics display region; and in response to a selection instruction input by a user through the selection operation control, the distribution statistic result of the physiological parameter to be displayed in the distribution statistics display region and the value of the pre-set time duration are determined. For example, the selection operation control can provide options of multiple parameter-value partitions, where the user can select a certain parameter-value partition, and only the distribution statistic result corresponding to the selected parameter-value partition is displayed in the distribution statistics display region. The selection operation control may also include a time input sub-control to enable the user to input the value of the pre-set time duration. Still further, a parameter selection operation control for the physiological parameter may also be provided, and in response to the selection instruction input by the user through the control, the physiological parameter(s) to be monitored is/are determined.

The distribution statistic result of the data within the pre-set time duration may be displayed either in the form of text or in the form of a distribution statistic chart. The specific display mode can refer to the detailed description below, which will not be repeated here.

It can be seen from the above technical solution that this disclosure provides a monitoring information display method, which method can acquire either the measurement data of the physiological parameter of the monitored object or the data within the pre-set time duration of the physiological parameter, perform statistics on the data within the pre-set time duration according to the parameter-value partition to obtain the distribution statistic result, and display the measurement data and the distribution statistic result in different regions of the monitoring interface. From the monitoring interface, the medical personnel can either view the current situation of the physiological parameter of the monitored object or view the distribution statistics of the physiological parameter, and the information of the two aspects can provide more abundant information for the medical personnel to determine the physiological state of the monitored object. Also, the data within the pre-set time duration are classified and displayed according to different parameter-value partitions, and the medical personnel can conveniently determine the changes in the physiological parameter of the monitored object through comparison.

Several specific implementation forms of the monitoring interface will be illustrated below, and in different monitoring interfaces, the measurement data of the physiological parameter and the distribution statistic result are displayed in different forms.

In one implementation form, after the distribution statistic result is obtained, a distribution statistic chart/table may also be generated according to the parameter-value partition and the distribution statistic result, and the distribution statistic chart/table is displayed in the distribution statistics display region. The distribution statistic chart/table may include a statistic chart such as a pie chart or a histogram, and may also include a statistic table.

One specific implementation form of the distribution statistic chart/table includes a histogram, specifically a histogram with the parameter-value partition and the distribution statistic result as two coordinate axes. The histogram can also be referred to as a statistic histogram.

In this case, one form of the parameter-value partition is the numerical interval of the physiological parameter, and the distribution statistic result is the number or the ratio of two numbers of the data within the pre-set time duration, falling into the respective numerical intervals, in the data of the physiological sign parameter within the pre-set time duration. Alternatively, the parameter-value partition and the distribution statistic result may also be applied to other forms of distribution statistic chart/table.

In this case, the data of the physiological parameter within the pre-set time duration include the data obtained by means of continuous measurement or discontinuous measurement between any two time points. For example, they are the data obtained by means of continuous measurement or discontinuous measurement between a current time point and a historical time point. Alternatively, such data within the pre-set time duration may also be applied to other forms of distribution statistics.

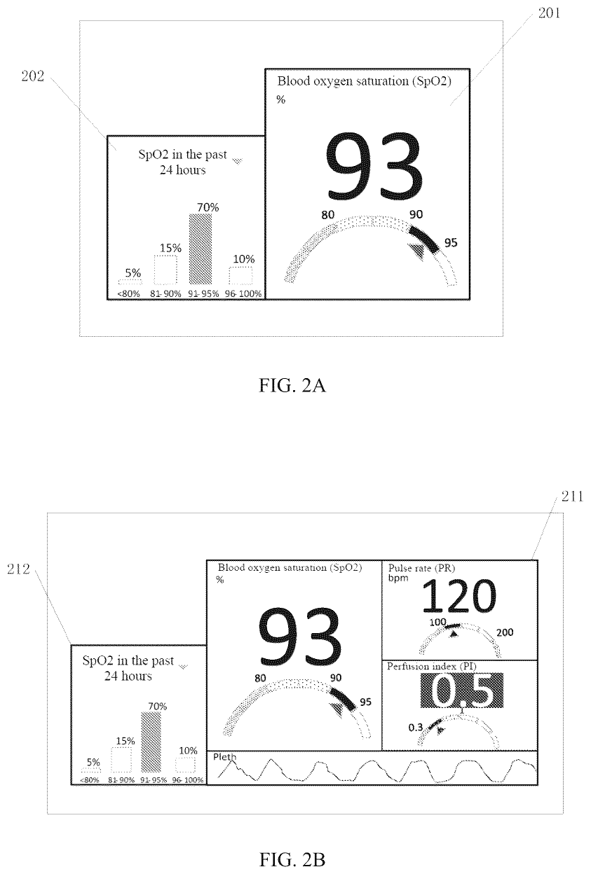

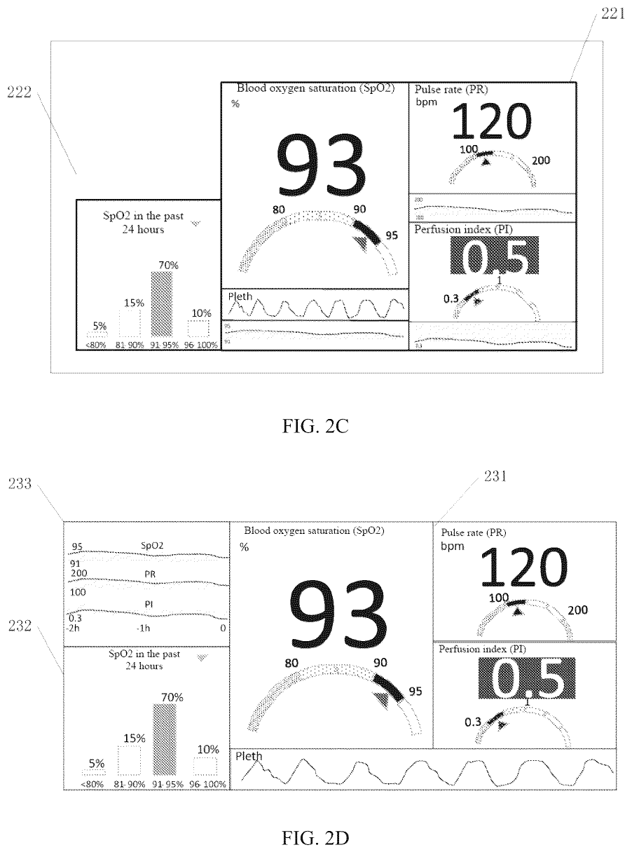

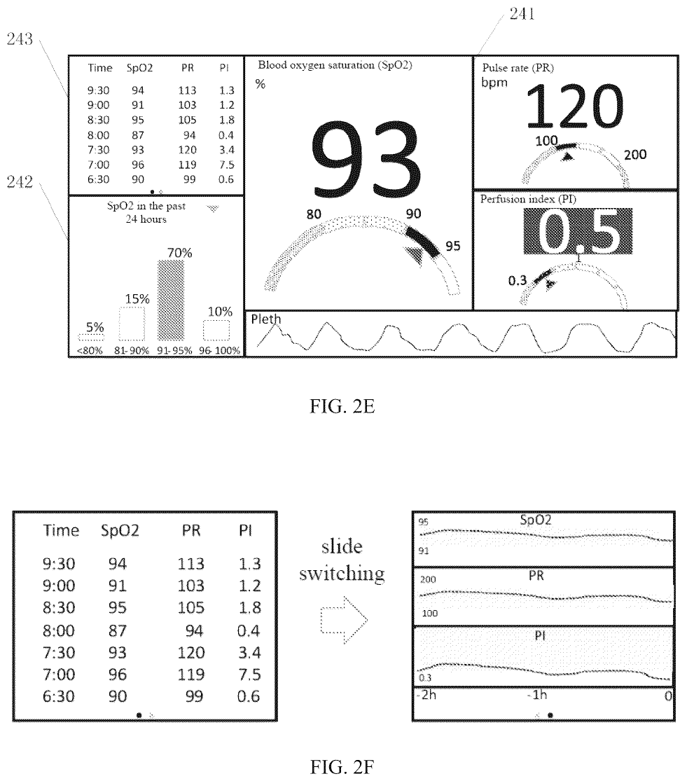

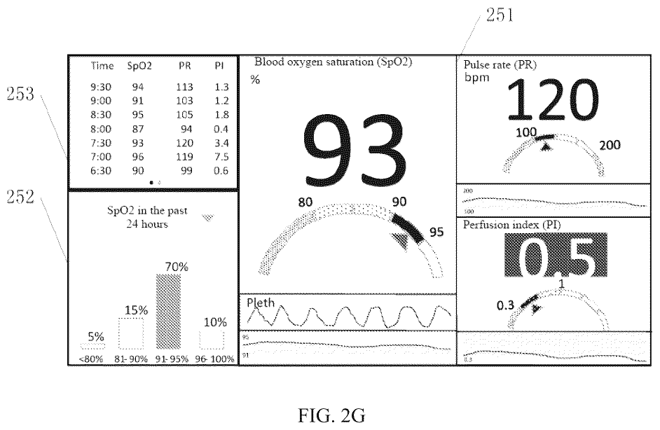

FIG. 2A shows an example of the monitoring interface. As shown in FIG. 2A, the physiological parameter displayed at the monitoring interface is blood oxygen saturation (SpO2). The monitoring interface contains the measurement data display region 201 and the distribution statistics display region 202, in which the measurement data display region 201 displays that the measurement data of the blood oxygen saturation is 93%, the distribution statistics display region 202 displays a histogram of the blood oxygen saturation, and the histogram is obtained by means of segmented statistics on the data of blood oxygen saturation within the pre-set time duration. It can be seen from the histogram that the data within the pre-set time duration are the data measured within 24 hours (h) before the current time point. It should be noted that the time duration can be selected and set by the user; for example, such as a downward triangle mark is provided in FIG. 2A, and the user can select the time duration provided therefor by means of clicking the triangle mark.

The horizontal coordinates of the histogram are four parameter intervals of the blood oxygen saturation, which are respectively [0-80%], [81%-90%], [91%-95%] and [96%-100%]. The vertical coordinate of the histogram is the ratio of the data of the blood oxygen saturation within each parameter interval to the total data within the pre-set time duration. By means of viewing the histogram, the medical personnel can ascertain that the blood oxygen saturation statistic (SpO2 statistic) of the monitored object in the past 24 hours includes: the number of values of blood oxygen saturation less than 80% accounts for 5% of the total, the number of values of blood oxygen saturation greater than 81% and less than 90% accounts for 15% of the total, the number of values of blood oxygen saturation greater than 91% and less than 95% accounts for 70% of the total, and the number of values of blood oxygen saturation greater than 96% and less than 100% accounts for 10% of the total.

It should be noted that, in a practical application, it is not limited to only generate the histogram for the blood oxygen saturation, and on the basis of including the histogram for the blood oxygen saturation, it is also possible to generate a histogram for other types of physiological parameters. Alternatively, only histograms for other types of physiological parameters without including blood oxygen saturation are generated.

In addition, in the monitoring interface shown in FIG. 2A, the measurement data displayed in the measurement data display region 201 are not limited to blood oxygen saturation, and may also be other types of physiological parameters. Alternatively, the measurement data display region is divided into multiple sub-regions, where the measurement data of the blood oxygen saturation is displayed in a certain sub-region, and the measurement data of other types of physiological parameters are displayed in other regions.

Another specific implementation form of the distribution statistic chart/table includes a statistic table, specifically a statistic table with the parameter-value partition as a header and with the statistic result as a table content. Similar to the above histogram, the statistic table shows how the distribution statistic result is under each parameter-value partition. Different from the above histogram, instead of configuring the parameter-value partition and the distribution statistic result as the horizontal coordinates and the vertical coordinates, the statistic table uses the parameter-value partition as the header, i.e., the column attribute, and uses the distribution statistic result as the recording content in the table.

Taking the histogram in FIG. 2A as an example, the parameter intervals can be used as the column attributes, the numerical value corresponding to each column attribute is the quantity proportion corresponding to the parameter interval, and each quantity proportion corresponding to each parameter interval constitutes one record in the statistic table. From this record, it can be seen how the distribution statistic results are under different parameter-value partitions.

It can be seen from the above example that this display mode of the distribution statistic chart/table is relatively standard and simple, and is convenient for the medical personnel to view and ascertain.

It should be noted that the distribution statistic result may correspond to multiple parameter-value partitions, and certain parameter-value partition(s) may have special significance as compared to other parameter-value partitions. The special meaning can specifically mean that it can provide guidance significance for the medical personnel to determine the physiological state of the monitored object. For ease of description, the parameter-value partition with this special significance can be referred to as the target parameter-value partition. The target parameter-value partition may be a parameter-value partition corresponding to the normal physiological state, or a parameter-value partition corresponding to the abnormal physiological state, or a parameter-value partition desired to be reached after treatment by medical means (where the parameter-value partition desired to be reached after treatment can be referred to as a target treatment region, and the other parameter-value partitions can be referred to as non-target treatment regions). Which parameter-value partition(s) the target parameter-value partition is/are may be preset by the system, or may be selected and set by the user.

In order to differentiate the target parameter-value partitions, a display pattern different from other parameter-value partitions is used to display the target parameter-value partition and/or the distribution statistic result corresponding to the target parameter-value partition. Taking the blood oxygen saturation statistic shown in FIG. 2A as an example, if the target treatment region is [91%-95%], the corresponding parameter interval is filled with background color. Alternatively, the differentiation of the display modes is not limited to color, and may also be performed by means of adding icons or symbols.

Alternatively, in order to facilitate the medical personnel to count the abnormal numerical values or normal numerical values contained in the statistic result, a target condition can be pre-set in the parameter-value partition. The distribution statistic result corresponding to the target condition is displayed in different display modes. Specifically, the target condition may indicate that the physiological parameter is in a normal or abnormal range so as to remind the medical personnel that the monitored object is in a normal or abnormal state. Of course, the target condition may also be a self-defined range desired to be considered by the medical personnel.

Therefore, the method for displaying monitoring information may also include: determining one target condition from the at least one parameter-value partition according to setup information; and determining, for the distribution statistic result corresponding to the target condition, a display pattern different from that for other distribution statistic results. As such, when the distribution statistic result is displayed, the distribution statistic result can be displayed, based on the display pattern, in the distribution statistics display region of the monitoring interface.

Further, in order to provide the medical personnel more viewing information, the method for displaying monitoring information can further display a trend chart and/or a waveform chart corresponding to the physiological parameter on the basis of displaying the measurement data of the physiological parameter.

Specifically, the corresponding waveform chart and/or the trend chart is/are generated according to the data of the at least one physiological parameter within the pre-set time duration, and the waveform chart and/or the trend chart is/are displayed in the measurement data display region. For example, for the measurement of blood oxygen saturation, a tracing wave with respect to the blood oxygen saturation (a waveform chart) or a blood oxygen saturation trend chart (the chart depicted based on the blood oxygen saturation values obtained by means of continuous measurement within a time period) can be displayed in the measurement data display region. Specifically, the measurement data and the waveform chart/trend chart corresponding to the same physiological parameter can be displayed close together.

The presentation charts (i.e., the waveform charts and/or the trend charts) corresponding to different types of physiological parameters are in different forms. In order to display the presentation chart of the physiological parameter in the measurement data display region, the measurement data display region can be divided into multiple sub-regions, of which some display the measurement data of the physiological parameter, and some display the presentation chart of the physiological parameter.

FIG. 2B shows yet another example of the monitoring interface. As shown in FIG. 2B, the physiological parameters involved in the monitoring interface include blood oxygen saturation (SpO2), pulse rate (PR) and perfusion index (PI). The monitoring interface contains a measurement data display region 211 and a distribution statistics display region 212. The measurement data display region 211 contains the measurement data of three physiological parameters, including blood oxygen saturation (SpO2), pulse rate (PR) and perfusion index (PI), which are respectively 93%, 120 bpm and 0.5. It should be noted that since the perfusion index, which is 0.5, exceeds the alarm threshold, this measurement data is displayed in an alarm pattern.

As shown in FIG. 2B, the alarm pattern includes displaying in an inverse color for the numerical value, and adding a background color. The content displayed in the distribution statistics display region 212 is the statistic histogram of the blood oxygen saturation.

In addition to the measurement data of the three physiological parameters mentioned above, the measurement data display region 211 further contains a plethysmogram (Pleth) related to the blood oxygen saturation. The measurement data display region can be divided into four sub-regions, in which three sub-regions are used to respectively display the measurement data of the three physiological parameters mentioned above, and the other sub-region is used to display the plethysmogram (Pleth).

Alternatively, it is possible to acquire the presentation charts of multiple physiological parameters, and the presentation charts of different physiological parameters can be respectively displayed in the sub-regions where the measurement data of the physiological parameter are located.

FIG. 2C shows yet another example of the monitoring interface. As shown in FIG. 2C, the monitoring interface contains a measurement data display region 221 and a distribution statistics display region 222. The content displayed in the measurement data display region 221 includes the measurement data of blood oxygen saturation 93%, the measurement data of pulse rate (PR) 120 bpm, the measurement data of perfusion index (PI) 0.5, and the content displayed in the distribution statistics display region 222 is a statistic histogram of the blood oxygen saturation.

It should be noted that, in addition to the measurement data of such three physiological parameters including blood oxygen saturation (SpO2), pulse rate (PR) and perfusion index (PI), the measurement data display region 221 further contains the presentation charts of the three physiological parameters. Specifically, the presentation charts include the plethysmogram (Pleth), a trend chart of the blood oxygen saturation, a trend chart of pulse rate (PR), and a trend chart of perfusion index (PI).

The trend chart may contain the normal parameter value range of the physiological parameter, and this range can be marked with shading and numerical values. As shown in FIG. 2C, it can be seen from the numerical scale that the normal parameter value range of blood oxygen saturation (SpO2) is 91% to 95%, the normal parameter value range of pulse rate (PR) is 100 bpm to 200 bpm, and the normal parameter value range of perfusion index (PI) is 1.0 or above.

It should be noted that the distribution statistics display region 222 and other information contained in FIG. 2C can refer to the illustration in FIGS. 2A and 2B described above, which will not be repeated here.

In yet another implementation form, rather than being displayed in the measurement data display region, the trend chart may be displayed in a trend display region different from the measurement data display region and the distribution statistics display region. Specifically, the corresponding trend chart and/or trend table is/are generated according to the data of the at least one physiological parameter within the pre-set time duration; and the trend chart and/or the trend table of the at least one physiological parameter is/are displayed in the trend display region of the monitoring interface.

In addition to being divided into the measurement data display region and the distribution statistics display region, the monitoring interface may also be divided into a trend display region, which is used to display the trend chart. The trend chart is generated from the data of the physiological parameter within the pre-set time duration. It should be noted that, when there are multiple types of physiological parameters, there are correspondingly multiple trend charts. Different physiological parameters correspond to different trend charts, and the multiple trend charts can be arranged vertically, side by side, or in other manners. In addition, the trend chart/trend table may refresh, i.e., the trend chart/trend table displayed in the trend display region may refresh in response to a pre-set trigger condition. The trigger condition may be a time condition, i.e., the data within the pre-set time duration are obtained after refresh at pre-set intervals, and a new trend chart is generated and displayed according to the data within the pre-set time duration after refresh. The trigger condition may also be the determination of the update of the measurement data of the corresponding physiological parameter. For example, after the update of the measurement data of the corresponding physiological parameter is determined, the trend chart/trend table displayed in the trend display region synchronously refreshes. Alternatively, after the update of the measurement data of the corresponding physiological parameter is determined, the distribution statistic result displayed in the distribution statistics display region may also synchronously refresh.

FIG. 2D shows another example of the monitoring interface. As shown in FIG. 2D, the physiological parameters displayed at the monitoring interface include blood oxygen saturation (SpO2), pulse rate (PR) and perfusion index (PI). Specifically, the monitoring interface contains a measurement data display region 231, a distribution statistics display region 232 and a trend display region 233. The content displayed in the measurement data display region 221 includes the measurement data of blood oxygen saturation 93%, the measurement data of pulse rate (PR) 120 bpm and the measurement data of perfusion index (PI) 0.5, and the content displayed in the distribution statistics display region 232 is a statistic histogram of the blood oxygen saturation.

The trend display region 233 sequentially contains, from top to bottom, trend charts of three physiological parameters including blood oxygen saturation (SpO2), pulse rate (PR) and perfusion index (PI). Of course, the arrangement of the three trend charts is not limited thereto, and the vertical order can be arranged arbitrarily, or other arrangement directions can also be used.

In addition, the three trend charts are respectively generated from the data of the three physiological parameters within the pre-set time duration. It can be seen from −2 h (hour) and −1 h (hour) in the figure that the data within the pre-set time duration are the data measured within 2 hours before the current time point 0.

It should be noted that the information in other region contained in FIG. 2D can refer to the illustration in FIGS. 2A-2C described above, which will not be repeated here.

With regard to the trend table, the header of the trend table contains the parameter-value partitions, and the content records in the table indicate how the distribution statistic result is under a certain parameter-value partition.

FIG. 2E shows another example of the monitoring interface. As shown in FIG. 2E, the physiological parameters displayed at the monitoring interface include blood oxygen saturation (SpO2), pulse rate (PR) and perfusion index (PI). The monitoring interface contains a measurement data display region 241, a distribution statistics display region 242 and a trend display region 243. The content displayed in the measurement data display region 241 includes the measurement data of blood oxygen saturation 93%, the measurement data of pulse rate (PR) 120 bpm and the measurement data of perfusion index (PI) 0.5, and the content displayed in the distribution statistics display region 242 is a statistic histogram of the blood oxygen saturation.

The trend display region 243 displays the trend chart of blood oxygen saturation (SpO2), pulse rate (PR) and perfusion index (PI). The trend chart shows the parameter values of blood oxygen saturation (SpO2), pulse rate (PR) and perfusion index (PI) at multiple different historical time points. The time points (Time) are respectively 6:30, 7:00, 7:30, 8:00, 8:30, 9:00, and 9:30, and the time interval between the multiple historical time points is fixed, which is 30 minutes. Of course, the time interval can be other values, and is not limited thereto. It should be noted that the time points in FIG. 2E are sorted from top to bottom, and in turn are time points that are farther and farther from the current time point. Of course, the order of time points can be reversed, that is, time points that are getting closer and closer to the current time point from top to bottom.

Of course, the historical time point may also be other forms of time points in the past time period. It should be noted that historical time point may have the time point selection criteria. For example, as shown above in FIG. 2E, the time points include the integral time points of one hour and the time points of 30 minutes. The cut-off time point of the historical time points may be the time point that meets the above selection criteria and is closest to the current time point. For example, the current time point is 9:40, and the most recent historical time point acquired in FIG. 2E is 9:30.

During the execution of this method, the defined pre-set time duration can be changed with the change of time, so the data obtained within the pre-set time duration are changed, and further the displayed distribution statistic result can refresh with a refresh interval being set by the user. Taking FIG. 2E as an example, assuming that the current time point changes from 9:40 to 10:01, the data within the pre-set time duration of blood oxygen saturation (SpO2), pulse rate (PR) and perfusion index (PI) till 10:00 can be acquired and displayed in the first row. Accordingly, the data of blood oxygen saturation (SpO2), pulse rate (PR) and perfusion index (PI) at the time point 6:30 in the last row can be omitted from the display, or are displayed on another screen, or continue to be displayed.

The number of records displayed in the trend chart can be either a preset fixed value or be set to the numerical value indicated by the instruction according to the user's setup instruction. If multiple statistic records cannot be displayed at the same time, they can be displayed on multiple screens, and the user can view other statistic records contained in other screens through touch operations.

It should be noted that the trend chart and the trend table are two different trend representations, and it is possible to preset which trend representation is fixedly displayed in the trend display region. Alternatively, the trend chart and the trend table can be mutually switched, and the triggering instruction of switching can be implemented by the user. Specifically, in response to an instruction input by the user, the trend chart or the trend table in the trend display region can be switched therebetween.

In one example, the monitoring device may be provided with a button, such as a physical button or a virtual button displayed on the display screen, and the user can select whether a trend chart or a trend table is to be displayed by means of triggering the button.

In another example, in the case that the display screen of the monitoring device has a touch function, the way to input instructions can be a sliding operation on the display screen, such as sliding left or right, or sliding up or down. In order to facilitate the user to input accurate switching and sliding instructions, the trend display region may also contain a prompt icon to prompt the user the direction of sliding so as to trigger the display of another trend representation.

As shown in FIG. 2F, the display screen of the monitoring device contains a monitoring interface. The trend display region of the monitoring interface currently displays a trend table. The bottom of the trend table contains two circular icons. The circular icon on the left is darker, and the circular icon on the right is lighter, which indicates that the current display is the trend representation on the left, and sliding to the right is needed to trigger the display of another trend representation. Based on the prompt, the user triggers a sliding operation to the right on the display screen, and the content displayed in the trend display region can be switched from a trend table to a trend chart. At the same time, the circular icons change, in which the circular icon on the left is lighter, and the circular icon on the right is darker. Of course, the switching can be performed in the opposite direction to switch the trend chart to a trend table.

Assuming that the trend charts or the trend tables are respectively in the patterns in FIGS. 2D and 2E, based on the above operation of switching a trend table to a trend chart, the entire monitoring interface can be switched from the pattern in FIG. 2E to the pattern in FIG. 2D.

It should be noted that the switching operation mode can not only facilitate the medical personnel to view different forms of trend representations, but also save the area occupied by the trend display region in the monitoring interface.

In the case that the monitoring interface contains a trend table or a trend chart, it is also possible to make a prompt for the abnormal situation in the trend table or the trend chart. Specifically, the parameter value of the physiological parameter in the trend table is monitored, and if the parameter value exceeds the corresponding parameter threshold, the parameter value is displayed based on a preset display pattern; or the parameter value of the physiological parameter in the trend table is monitored, and if alarm information associated with the parameter value is monitored, the parameter value is displayed based on a preset display pattern; or the trend table is displayed in a display pattern that can differentiate the normal and abnormal parameter values of the physiological parameter in the trend table.

The alarm information may be an alarm condition defined according to an abnormal physiological state, and when the parameter value of the physiological parameter triggers the alarm information, it is necessary to make a prompt for this parameter value. For example, the alarm information related to the blood oxygen saturation may include low blood oxygen saturation, and when a certain parameter value of the blood oxygen saturation triggers the low blood oxygen saturation alarm, it is necessary to make a prompt for this parameter value. For another example, the alarm information related to the pulse rate may include bradycardia, extreme bradycardia, or excessive slow heartbeat, and when a certain parameter value of the pulse rate triggers any of the above alarm information, it is needed to make a prompt for this pulse rate.

Taking FIG. 2E as an example, if the physiological parameter in a certain record in the trend table exceeds the corresponding parameter threshold or triggers the associated alarm information, the displayed font color can be set to be different from the font color of other records, or a background color is added for this record. Likewise, the curves corresponding to normal and abnormal parameter values of the physiological parameter in the trend chart can be differentiated by different colors. Of course, the differentiation of the display patterns is not limited to colors, and may also be performed in various forms, such as by adding background patterns, icons and/or symbols.

It should be noted that the various forms of representations above can be displayed in any combination. For example, FIG. 2A, 2B or 2C can be combined with a trend table or a trend chart. FIG. 2G shows yet another example of the monitoring interface. As shown in FIG. 2G, the monitoring interface contains a measurement data display region 251, a distribution statistics display region 252 and a trend display region 253. The measurement data display region 251 contains the measurement data of three physiological parameters including blood oxygen saturation (SpO2), pulse rate (PR) and perfusion index (PI). Specifically, the measurement data of blood oxygen saturation (SpO2) is 93%, the measurement data of pulse rate (PR) is 120 bpm (beat per minute), and the measurement data of perfusion index (PI) is 0.5. The content displayed in the distribution statistics display region 252 is the statistic histogram of the blood oxygen saturation. The content displayed in the trend display region 253 includes the trend tables of the blood oxygen saturation (SpO2), the pulse rate (PR) and the perfusion index (PI).

It should be noted that other information contained in FIG. 2G can refer to the above illustration, which will not be repeated here. In addition, in the figures related to the monitoring interface herein, the measurement data display region where the measurement data are located further contains a state indicator, which contains multiple parameter-value partitions and one indicator mark that is used to indicate the parameter-value partition to which the measurement data belong.

An apparatus embodiment related to the method for displaying monitoring information will be described below, and the description regarding the apparatus can refer to the method embodiment part described above, which will not be repeated below.

The embodiments of this disclosure further provide another method for displaying monitoring information. It should be noted that some specific implementations of the method for displaying monitoring information are the same as or similar to the method for displaying monitoring information provided in the above embodiments. The following is only a brief description of this another method for displaying monitoring information. It should be understood that the related technical solutions can be also applied here by those skilled persons in the art on the basis of the above description.

The method for displaying monitoring information includes obtaining data of at least one physiological parameter of a monitored object within a pre-set time duration, where the data of the at least one physiological parameter of the monitored object within the pre-set time duration is obtained from the monitored object by means of at least one physiological sensor.

The method for displaying monitoring information then includes performing distribution statistics on the data within the pre-set time duration based on at least one parameter-value partition, and determining a distribution statistic result corresponding to the parameter-value partition, where the parameter-value partition represents a numerical interval of the physiological parameter, and the numerical internal is determined based on one or more of an alarm threshold, a baseline range, and a treatment target range corresponding to the physiological parameter.

The method for displaying monitoring information further includes providing a monitoring interface, generating a distribution statistics display region at the monitoring interface, and displaying the distribution statistic result in the distribution statistics display region of the monitoring interface.

In an embodiment, the alarm thresholds may be numerical value ranges respectively corresponding to levels of critical alarm, intermediate alarm, etc.; the baseline range can refer to the normal numerical range of the physiological parameters of the current monitored object; and the treatment target range may refer to the numerical value range that the physiological parameter of the monitored object is enabled to reach after the monitored object is treated by the medical personnel.

In this embodiment, the distribution statistic result is not obtained only by dividing the physiological data of the monitored object; instead, the distribution statistic result is obtained by the distribution statistics on the data within the pre-set time duration according to the numerical interval which is determined based on the alarm threshold, the baseline range or the treatment target range. The distribution statistic result obtained in this way can allow the medical personnel to intuitively ascertain the distribution of the data of the physiological parameter of the monitored object within a time period in the alarm threshold range, the baseline range and the treatment target range so as to facilitate ascertaining the physiological state of the monitored object. Simple division of numerical interval will provide messy data. Even if the medical personnel see the distribution result, they cannot intuitively ascertain the physiological state of the monitored object associated with these distribution result. However, the method for displaying monitoring information provided in this embodiment solves this problem well.

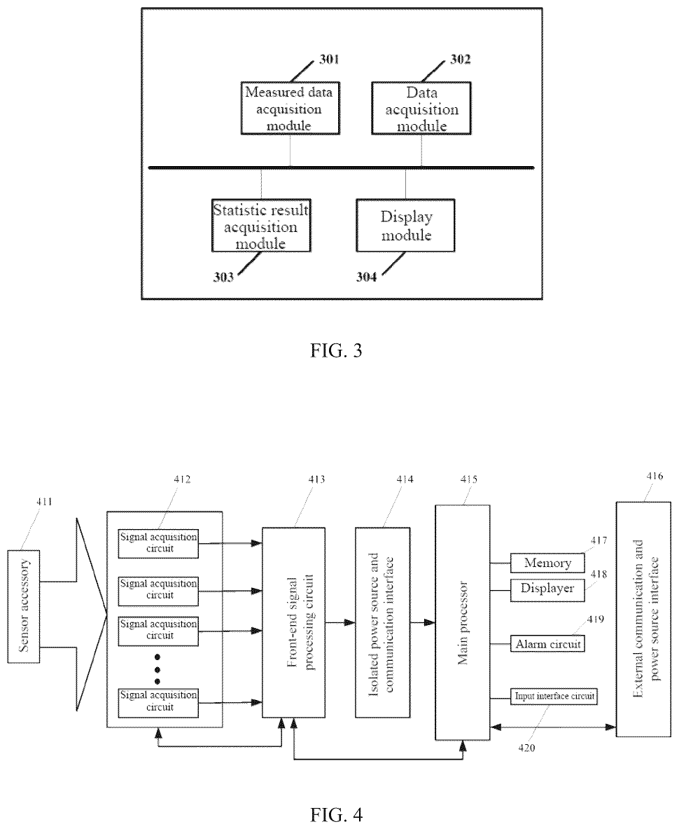

FIG. 3 shows a schematic structural diagram of an apparatus for displaying monitoring information provided in this disclosure. As shown in FIG. 3, the apparatus for displaying monitoring information may specifically include a measured data acquisition module 301, a data acquisition module 302, a statistic result acquisition module 303, and a display module 304.

Furthermore, although current monitors can display measurement data of some physiological parameters of a patient, the display mode is relatively simple, where only the numerical value of the measurement data or a trend chart and/or a waveform chart corresponding to the physiological parameter is displayed. The medical personnel can only determine whether the current measurement data fall within an expected target range and whether it falls within the target range most of the time by themselves (a treatment target range is often not equal to an alarm range, and in the prior art, there was no explicit indication of whether the measurement data is within the target range). The display of whether the current measurement data falls within the expected target range is not very intuitive, and thus cannot obviously provide corresponding diagnosis and treatment reference information for the medical personnel and cannot meet the monitoring needs of the medical personnel very well.

This disclosure further provides a method for displaying a physiological parameter, which can improve the richness of the displayed parameter content and help the medical personnel to ascertain the current physiological state of the monitored object.

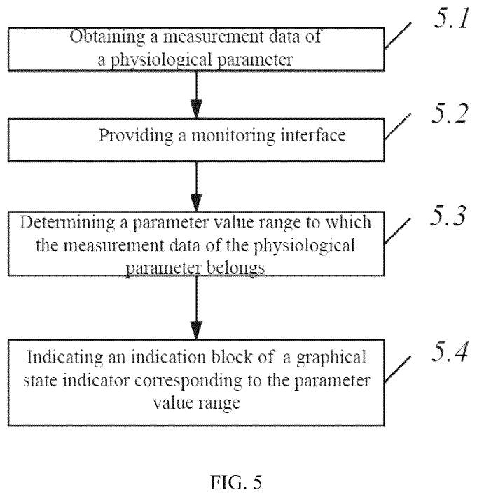

FIG. 5 is a flow chart of the method for displaying the physiological parameter. As shown in FIG. 5, the method can specifically include steps 5.1-5.4.

At step 5.1: a measurement data of a physiological parameter is obtained.

Specifically, a measurement data of at least one physiological parameter of a monitored object is obtained, where the measurement data of the physiological parameter is obtained from the monitored object by means of at least one sensor. The physiological parameter may be a parameter of any physiological sign of the monitored object, which for example can be heart rate, pulse rate, blood oxygen saturation, respiratory rate, etc.

The measurement data of the physiological parameter collected by the sensor can be sent to a storage module of the monitoring system for storage, and these basic data can be used as basic data for display and analysis. That is to say, for the measurement data of the physiological parameter, the measurement data of the physiological parameter can be stored into the storage module of the monitoring system by means of the sensor, and then obtained from the storage module in this disclosure. Of course, the measurement data of the physiological parameter may also be directly obtained from the sensor.

Therefore, the method for displaying the physiological parameter provided in this disclosure can be applied not only to bedside devices, but also to central stations. When applied to a bedside device, the measurement data of the physiological parameter is obtained by means of various physiological parameter accessory devices; and when applied to a central station, the central station obtains the data of the physiological parameter from the bedside device via a network.

At step 5.2, a monitoring interface is provided.

Specifically, the monitoring interface includes a state indication region that displays at least one graphical state indicator including multiple indication blocks. Each of the indication blocks respectively corresponds to a parameter value range of the physiological parameter, and the multiple indication blocks are displayed in the state indication region in an orderly arrangement according to the numerical value of the physiological parameter.

The state indication region on the monitoring interface may be a region embedded in the monitoring interface, or may be a window region suspended on the monitoring interface. The position attribute, shape attribute, display attribute, state attribute, etc. of the state indication region can all be adjusted. For example, the position attribute of the state indication region refers to the display position of the state indication region on the monitoring interface; the shape attribute of the state indication region includes the pattern, the size, etc. of the shape (for example, the shape may be various shapes, such as rectangle, circle and heart shape); the display attribute of the state indication region refers to attribute information, such as color, brightness and contrast, of all or part of the region; and the state attribute of the state indication region includes attributes of being displayed or not displayed, being embedded in or suspended on the monitoring interface, etc.

The monitoring interface includes a chart containing multiple indication blocks, where different indication blocks correspond to different parameter value ranges of the physiological parameter, and different parameter value ranges can represent different physiological states of the monitored object. A visible chart has an intuitive indication of the physiological state, and for ease of description, the chart can be referred to as the graphical state indicator. The indication blocks are arranged in an orderly manner according to the numerical value of the physiological parameter, such as according to the order from left to right or from top to bottom, and the parameter value of the physiological parameter gradually increases along the orderly direction.

It should be noted that the number of the graphical state indicators is not limited to one, and may also be more than one. In the case where there are multiple graphical state indicators, different graphical state indicators correspond to different physiological parameters.

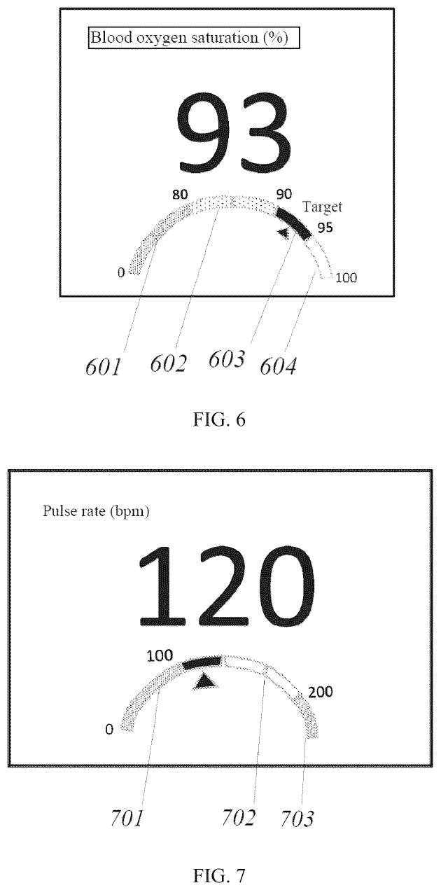

FIGS. 6-8 show the graphical state indicators respectively corresponding to blood oxygen saturation, pulse rate and perfusion index.

As shown in FIG. 6, the graphical state indicator for blood oxygen saturation (SpO2) includes four indication blocks. The indication block 601 corresponds to the parameter value range in which the blood oxygen saturation value is 0 to 80%, which is used to represent a high-severity state; the indication block 602 corresponds to the parameter value range in which the blood oxygen saturation value is 80%-90%, which is used to represent a moderate-severity state; the indication block 603 corresponds to the parameter value range in which the blood oxygen saturation value is 90%-95%, which is used to represent a treatment target state of blood oxygen saturation; and the indication block 604 corresponds to the parameter value range in which the blood oxygen saturation value is 95%-100%, which is used to represent a normal blood oxygen saturation state. Of course, four indication blocks are described above. In fact, each of the indication blocks can be further subdivided into multiple indication sub-blocks.

As shown in FIG. 7, the graphical state indicator for pulse rate (PR) includes three indication blocks. The indication block 701 corresponds to the parameter value range in which the pulse rate value is 0-100, which can represent a low pulse rate state; the indication block 702 corresponds to the parameter value range in which the pulse rate value is 100-200, which can represent a physiological alarm range of pulse rate; and the indication block 703 corresponds to the parameter value range in which the pulse rate value is greater than 200, which can represent a state where the pulse rate is too fast. The unit of pulse rate value is beat per minute (bpm). Of course, three indication blocks are described above. In fact, each of the indication blocks can be further subdivided into multiple indication sub-blocks.

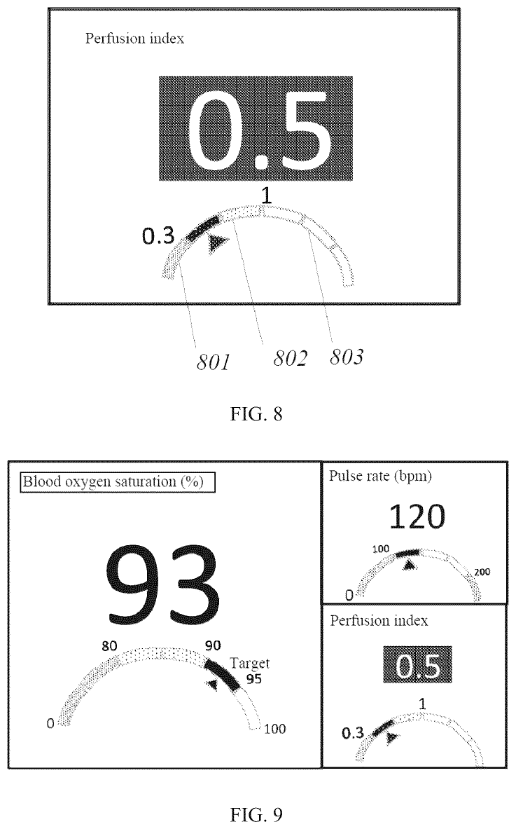

As shown in FIG. 8, the graphical state indicator for perfusion index (PI) includes three indication blocks. The indication block 801 corresponds to the parameter value range in which the perfusion index value is 0-0.3, which can represent the state where the patient's weak perfusion is severe, and since the weak perfusion is too low, the measured blood oxygen saturation value may not be accurate enough at this time; the indication block 802 corresponds to the parameter value range in which the perfusion index value is 0.3-1, which can represent that the patient has a weak perfusion problem currently; and the indication block 803 corresponds to the parameter value range in which the perfusion index value is greater than 1, which can represent that the patient has good perfusion condition currently. Of course, three indication blocks are described above. In fact, each of the indication blocks can be further subdivided into multiple indication sub-blocks.

It should be noted that for different physiological parameters, the parameter value ranges corresponding to the indication blocks are different, and can be set according to the clinical guidance significance. In addition, in order to help the medical personnel to ascertain the range of parameter-value partitions in the graphical state indicator, as shown in FIGS. 6-8, the graphical state indicator includes critical values of the parameter-value partition.

In the case where there are multiple physiological parameters, the graphical state indicators of the multiple physiological parameters can be simultaneously displayed on the monitoring interface. As shown in FIG. 9, the monitoring interface includes graphical state indicators for three physiological parameters, including blood oxygen saturation (SpO2), pulse rate (PR) and perfusion index (PI).

At step 1.3, the parameter value range to which the measurement data of the physiological parameter belongs is determined.

Specifically, monitoring data that comprises the measurement data of the physiological parameter can be generated by monitoring the target object. The graphical state indicator of the physiological parameter includes multiple parameter value ranges, and according to the numerical value of the measurement data of the physiological parameter, the parameter value range to which the measurement data belongs is determined. The measurement data of the physiological parameter may specifically be the currently measured parameter value, i.e., the real-time measurement data.

For example, the physiological parameter is blood oxygen saturation, and as shown in FIG. 6, the graphical state indicator for blood oxygen saturation includes four parameter value ranges. Assuming that the measurement data of the blood oxygen saturation of certain monitored object is 93%, it can be seen from the four parameter value ranges divided in FIG. 6 that the parameter value range to which the measurement data belongs is the parameter value range corresponding to 90%-95%.

At step 1.4, display and output operations is performed in the indication block of the graphical state indicator corresponding to the parameter value range to which the measurement data belongs.

The graphical state indicator includes multiple indication blocks, and different indication blocks correspond to different parameter value ranges. After the parameter value range to which the measurement data belongs is determined, the indication block corresponding to the parameter value range is indicated. Different display patterns are used to differentiate the display of the multiple indication blocks of the graphical state indicator. The display mode may include differentiating by color, highlighting, adding indication marks, etc., and the specific indication mode will be described in detail below in conjunction with the accompanying drawings, which will not be repeated here. Of course, the display mode may also be other modes, as long as it can be differentiated from other parameter-value partitions.

Different parameter value ranges represent different physiological states, and the indication about which parameter value range the measurement data belongs to can indicate the medical personnel what physiological state the monitored object has at the time point when the measurement data is collected. Still taking FIG. 6 as an example, in the graphical state indicator, the range of the parameter value range of 90%-95% represents the treatment target state of the blood oxygen saturation, and if the parameter value range to which the measurement data belongs is this target range, it represents that the physiological state of the monitored object has reached the desired target state of the treatment.

It can be seen from the above technical solutions that this disclosure provides a method for displaying the physiological parameter. The method can provide, on the monitoring interface, the graphical state indicator for the physiological parameter, where the graphical state indicator includes multiple indication blocks, and the parameter-value partitions corresponding to different indication blocks correspond to different physiological states; the method can also obtain a measurement data of the physiological parameter of the monitored object, determine the parameter value range to which the measurement data belongs, and indicate, in the graphical state indicator, the indication block corresponding to the parameter value range. In this way, the medical personnel can ascertain the current physiological state of the monitored object by means of viewing the indicated indication block.

With the development of detailed monitoring, the evaluation of the measurement data in clinical practice is no longer limited to a single state of whether the measurement data exceeds an alarm threshold. Instead, the medical personnel hope to be able to perform more patient status grading for the measurement data. The graphical state indicator provided in this disclosure includes multiple parameter value ranges, different parameter value ranges correspond to different physiological states, and the medical personnel can perform detailed monitoring for a variety of different physiological states of the monitored object by means of viewing the graphical state indicator.

It will specifically illustrate how to indicate, in the graphical state indicator, the indication block corresponding to the measurement data.

In an embodiment, each of the multiple indication blocks of the graphical state indicator is provided with an initial brightness value and a preset brightness value. The initial brightness value refers to a brightness value which represents that the measurement data does not correspond to the indication block; and the preset brightness value refers to a brightness value which represents that the measurement data corresponds to the indication block.

Therefore, in order to indicate the indication block corresponding to the measurement data, the display brightness of the indication block corresponding to the parameter value range to which the measurement data belongs can be increased from the initial brightness value to the preset brightness value in the graphical state indicator. In order to achieve better indication effect, the initial brightness value is low brightness, and the preset brightness value is high brightness. That is to, the indication block corresponding to the parameter value range to which the measurement data belongs is highlighted.

The multiple indication blocks may have the same initial brightness values and the same preset brightness values. After the indication block corresponding to the measurement data is determined, only the indication block corresponding to the measurement data is displayed with brightness adjustment. Alternatively, the multiple indication blocks may also have different initial brightness values and different preset brightness values.

In an embodiment, each of the multiple indication blocks of the graphical state indicator is provided with an initial color and a preset color. The initial color value represents that the measurement data does not correspond to the indication block of the initial color value; and the preset color value represents that the measurement data corresponds to the indication block of the preset color value.

Therefore, in order to indicate the indication block corresponding to the measurement data, the display color of the indication block corresponding to the parameter value range to which the measurement data belongs can be increased from the initial color value to the preset color value in the graphical state indicator. In order to achieve better indication effect, the initial color value is light, and the preset color value is dark. That is, the indication block corresponding to the parameter value range to which the measurement data belongs is displayed in a darker color.

The multiple indication blocks may have the same initial color values and the same preset color values. After the indication block corresponding to the measurement data is determined, only the indication block corresponding to the measurement data is displayed in a darker color. Alternatively, the multiple indication blocks may also have different initial color values and different preset color values.

In some embodiments, it may be a combination of the above two embodiments.

In an embodiment, the multiple indication blocks of the graphical state indicator include a target indication block used to represent the expected state of the physiological parameter. Therefore, if the parameter value range to which the measurement data belongs is the parameter value range corresponding to the target indication block, the target indication block is displayed in a display and output mode differentiated from other indication blocks.

The expected state of the physiological parameter may include any one of the states such as a normal state, an abnormal state, and a treatment target state. The target indication block refers to the indication block representing a certain expected state in the multiple indication blocks. If the measurement data corresponds to the target indication block, the target indicate block is displayed and outputted differently from other indication blocks.

There may be many modes for differentiating the indication blocks. For example, if the display and output mode of the indication block is to adjust the display brightness, the mode for differentiating the indication blocks may refer to adjusting the display brightness value for the target indicate block to be larger or smaller. For another example, if the display and output mode of the indication block is to switch the display color, the mode for differentiating the indication blocks may refer to that the display color of the target indicate block is different from that of other indicate blocks after display color switching. For another example, the target indication block is elliptical in shape, and the non-target indication block is rectangular in shape. For another example, the target indication block is displayed with shading filling, and the non-target indication block is not displayed with shading filling. As another example, it may be a combination of the above modes or other differentiating modes.

The display and output mode will be illustrated below taking an example in which the expected state is a treatment target state.

The treatment target state represents the state that the monitored object needs to reach after the treatment means in a monitoring scenario. This state may be an expected treatment state generally recognized in the medical field for all monitored objects, or may also be an expected treatment state that is set for a specific monitored object and that the specific monitored object needs to reach in a specific monitoring scenario.

The parameter value range corresponding to the treatment target state may be referred to as the parameter value range of the treatment target value, that is to say, the parameter values belonging to the parameter value range are all the treatment target values. Also, in the multiple indication blocks, the indication block corresponding to the parameter value range of the treatment target value may be referred to as the treatment target indication block.

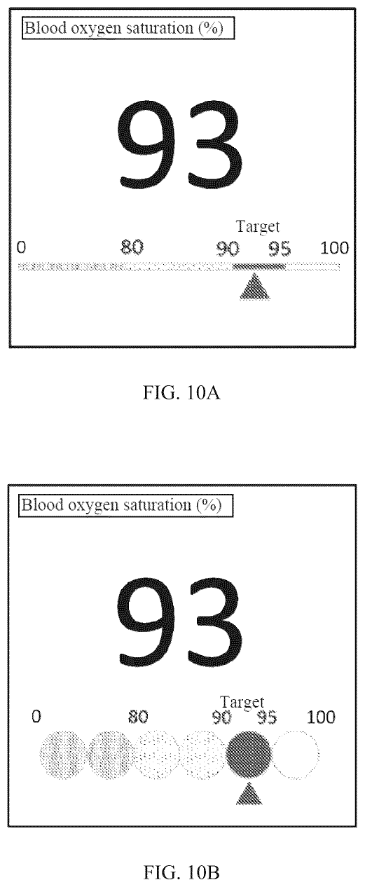

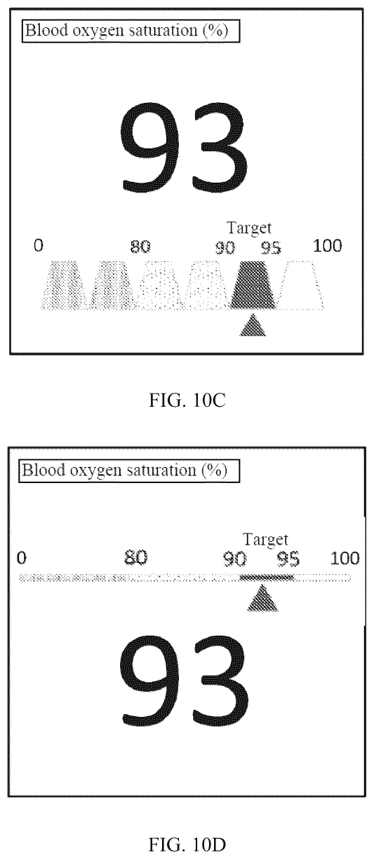

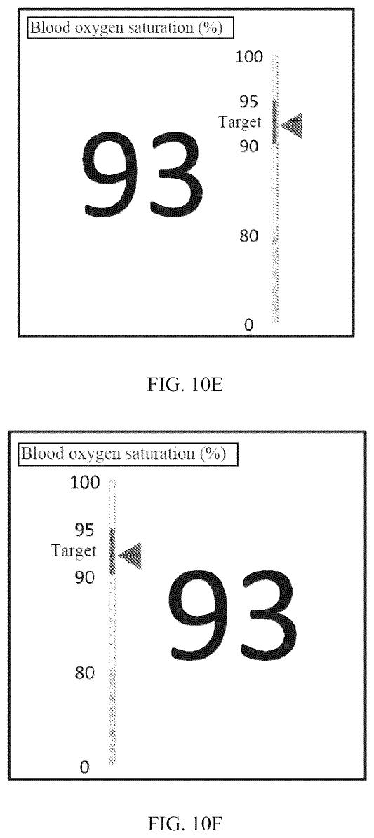

In order to make a prompt for the treatment target indication block, a prompt will be added in the graphical state indicator at the corresponding position of the treatment target indication block. The prompt may be in text form, such as “target” in Chinese (or “target” in English) in FIGS. 6 and 9 above and in FIGS. 10A-10F below. Alternatively, the prompt may also be a picture or a symbol, such as a picture or a symbol of a smiley face. Alternatively, the prompt may also be in other forms.

When the measurement data of the physiological parameter is determined to jump from a parameter value range to which the non-treatment-target value belongs to a parameter value range to which the treatment target value belongs, the treatment target indication block is displayed and outputted in a preset display pattern differentiated from an original display pattern.

The original display pattern represents a display pattern showing when the measurement data does not correspond to the treatment target indication block; and the preset display pattern represents a display pattern showing when the measurement data corresponds to the treatment target indication block. When the measurement data changes from the parameter value range to which the non-treatment-target value belongs to the parameter value range to which the treatment target value belongs, it represents that the measurement data changes from a state in which the measurement data does not correspond to the treatment target indication block to a state in which the measurement data corresponds to the treatment target indication block, and thus the display pattern needs to change from the original display pattern to the preset display pattern.

Of course, if the measurement data changes in an opposite way, i.e., changes from the parameter value range to which the treatment target value belongs to the parameter value range to which the non-treatment-target value belongs, the display pattern can change reversely by canceling the preset display pattern while displaying and outputting the treatment target indication block in the original display pattern.

The display pattern may include brightness value and/or display color. The specific change mode can be illustrated with reference to the first two embodiments in this part.

In an embodiment, in order to indicate the indication block corresponding to the parameter value range to which the measurement data belongs, an indication icon may be displayed at the position of the indication block corresponding to the parameter value range to which the measurement data belongs. The indication icon points to the indication block corresponding to the parameter value range to which the measurement data belongs.

The indication icon may be a cursor pointer, such as the triangular cursor pointer in FIGS. 6-10F. The position where the indication icon is added needs to be able to prompt the medical personnel, i.e., to indicate the medical personnel which indication block the measurement data corresponds to. For example, when the graphical state indicator is a horizontally placed straight-bar structure, the indication icon is added directly above or below the indication block. For another example, when the graphical state indicator has an arc structure, the indication icon is added at the central position of the arc of the indication block, etc.

It should be noted that the several embodiments mentioned above can be combined freely. For example, after the indication block corresponding to the measurement data is determined, the preset color of this indication block is highlighted, and the indication icon is added at the corresponding position of the indication block.

Further, in order to enable the medical personnel to ascertain the physiological state of the monitored object, the measurement data can be displayed in the state indication region of the monitoring interface, and/or the waveform chart and/or the trend chart corresponding to the physiological parameter can be displayed in the state indication region of the monitoring interface. For example, for the measurement of blood oxygen saturation, a tracing wave (a waveform chart) with respect to the blood oxygen saturation or a trend chart with respect to the blood oxygen saturation (the chart depicted based on the blood oxygen saturation values obtained by continuous measurement over a period of time) can be displayed in the state indication region.

The font and the size of the measurement data can be preset, or can be modified. For noticeable prompt, the font may be a relatively large-size font. That is, a large-font display mode is used.

The display position of the measurement data may be freely arranged, or may be set according to the position of the graphical state indicator. The graphical state indicator and the measurement data may be in the relative position of up and down, left and right, etc. The graphical state indicator may be located above the measurement data, or located below the measurement data; and the graphical state indicator may be located on the left of the measurement data, or on the right of the measurement data.

Seeing the monitoring interface shown in FIGS. 10D-10F below, the current measured value of the blood oxygen saturation 93% is respectively located below, at the left, and at the right of the graphical state indicator.

The physiological parameter may have an alarm threshold. If the measurement data exceeds the alarm threshold, the measurement data can be displayed according to a preset alarm prompt mode to prompt the medical personnel that the target physiological parameter of the monitored object is abnormal. The preset alarm prompt mode may include any one or more of: displaying in an inverse color, changing from a non-alarm color to an alarm color such as red, adding shading background, and flashing. As shown in FIGS. 8 and 9, the measured value of the perfusion index 0.5 exceeds the alarm threshold, and thus the numerical value 0.5 is inverted from black to white, and a gray shading background is added. Alternatively, the alarm prompt mode may also be other modes and is not limited thereto. It should be noted that the alarm threshold may include multiple alarm thresholds. Different alarm thresholds correspond to different inverse colors, and when an alarm threshold is exceeded, the color needs to be inverted to the corresponding color of the exceeded alarm threshold. For example, the inverse color of a high-level alarm threshold is red, and the inverse color of an intermediate-level alarm threshold is yellow.

The graphical state indicator is formed by connecting multiple indication blocks. Regardless of whether the indication blocks are of the same shape or different shapes and whether the shape is regular or irregular, the multiple indication blocks are connected in such a manner that forms a connection line, and the shape of the connection line will determine the overall shape of the graphical state indicator. For example, for the graphical state indicator in FIGS. 7-9, the connection lines of the multiple indication blocks are arc-shaped lines, and thus the overall structure of the formed graphical state indicator is arc-shaped.

Alternatively, the graphical state indicator is not limited to be arc-shaped, but may also be in another shape. FIGS. 10A-10C are other implementations of the graphical state indicator shown in FIG. 6. As shown in FIG. 10A, the connection line of each indication block in the graphical state indicator is in a straight-line shape, so it can be considered that the structure of this graphical state indicator is in a straight-bar or straight-line shape. It should be noted that the graphical state indicator can be placed horizontally, vertically or at an angle in the case of the straight-line shape.

It should be noted that, no matter what connection shape of the graphical state indicator, the indication block may be either in a long-bar shape or a line shape. In this case, the graphical status indicator having the arc-shaped structure is similar to an instrument panel. In addition, as shown in FIGS. 10B and 10C, the indication blocks in the graphical state indicator may also be respectively circular or trapezoidal in shape. Alternatively, the indication block may also be in other shapes, and is not limited to what is shown in the accompanying drawings.

It can be seen from the above drawings that the graphical state indicators of any form all include multiple indication blocks, and different indication blocks correspond to different parameter-value partitions of the physiological parameter. The change of the parameter value can reflect the state change of the monitored object in this physiological parameter. Quantitative changes will cause qualitative changes. A parameter value in a certain range may represent a state of the monitored object, such as a normal state, and a parameter value in another range may represent another state of the monitored object, such as an abnormal state. Therefore, parameter-value partitions of a physiological indicator can be set according to the clinical experience, and different parameter-value partitions correspond to different physiological states.

It can be understood that when there are more parameter-value partitions, more physiological states can be reflected, and thus the monitoring of the monitored object can become more detailed. For example, there may be two parameter-value partitions, respectively representing the normal state and the abnormal state. For another example, there may be three parameter-value partitions, respectively representing the normal state, the mildly abnormal state, and the severely abnormal state.

In the embodiment described above, the measurement data of the physiological parameter is displayed in the state indication region. In some embodiments, the monitoring interface may also include the measurement data display region as described in FIGS. 2A, and the measurement data display region and the state indication region are two adjacent regions with the common boundary. The measurement data are displayed in the measurement data display region.

Specifically, the measurement data display region and the state indication region are two independent regions, and the two regions are positioned adjacently. The adjacent relationship between the state indication region and the measurement data display region does not include a closed surrounding relationship, but may include a semi-closed surrounding relationship. More specifically, the measurement data display region may be located beyond the arc of the state indication region, such as directly above or at the upper left of the state indication region, that is to say, the state indication region does not surround the measurement data display region.

In addition, the boundary lines of the measurement data display region and the state indication region may be displayed together or hidden together, or one of them is displayed while the other is hidden.

The multiple indication blocks of the graphical state indicator are arranged in a straight-bar shape or a circular arc shape in an orderly manner, and a parameter pointer is displayed in the state indication region with one end thereof indicating the position of the indication block corresponding to the parameter value range to which the measurement data belongs. It can also be seen from the circular arc shape that the graphical state indicator is not a closed circle, which further indicates that the graphical state indicator does not completely surround the measurement data. The description regarding the parameter pointer can refer to the description of the indication icon mentioned above, which will not be repeated here.

When multiple indication blocks of the graphical state indicator are arranged in a circular arc shape in an orderly manner, one end of the parameter pointer can indicate the position of the indication block corresponding to the parameter value range to which the measurement data belongs, and the other end thereof can be displayed at the center position corresponding to the circular arc shape. Such a display effect is similar to the instrument panel, where the measurement data is the value in the instrument panel, one end of the pointer is at the center position of the instrument panel, and the other end of the pointer points to the measurement data in the instrument panel. It should be noted that because the state indication region does not surround the measurement data display region, the parameter pointer will have such a display pattern.

The multiple indication blocks of the graphical state indicator are arranged in a circular arc shape in an orderly manner. In some embodiments, the circular arc shape may be in a shape smaller than a semicircle, i.e., the central angle corresponding to the circular arc shape is less than 180 degrees. In this way, the height of the graphical state indicator can be limited, such that the measurement data can be displayed in a large-font display pattern to highlight the measurement data.

The large-font display pattern of the measurement data refers to the pattern that can affect the size of the region occupied by the measurement data, such as font, font size, and font bold. That is to say, in order to achieve the large-font display pattern, a preset large font, a preset large font size and font bold can be used for the measurement data. Through the large-font display pattern, the measurement data can be noticeably displayed to have a reminding effect for the measurement data.

In practical applications, in addition to viewing the current measured physiological state of the monitored object, it is also needed to ascertain the historical physiological state of the monitored object. The combination of such two can provide the medical personnel with a richer diagnosis basis. Therefore, historical values of the physiological parameter can be analyzed, and an analysis result is displayed on the monitoring interface.

In an analysis mode, the distribution statistics described above can be performed on the historical values. Specifically, the historical values of at least one physiological parameter of the monitored object within a preset time duration are first obtained. The distribution statistics is then performed on the historical values according to at least one parameter-value partition, and the distribution statistic result corresponding to the parameter-value partition is determined. After that, the distribution statistics display region is provided on the monitoring interface, and the distribution statistic result is displayed in the distribution statistics display region. It should be noted that the parameter-value partition here may be different from the parameter value range corresponding to the indication block in the above graphical state indicator.

It should be noted that the preset time duration corresponding to the historical value, the parameter-value partition to be displayed, and/or the physiological parameter to be displayed, may be either preset by the system or be independently selected and set by a user.

Specifically, in response to a selection instruction input by a user, the distribution statistic result of the historical values of the target physiological parameter selected by the user is displayed in the distribution statistics display region. The selection instruction represents the target physiological parameter that the user pays attention to, and the selection of the preset time duration corresponding to the historical values thereof.

As described above, the selection operation control can be provided on the monitoring interface for the user to input the selection instruction. For example, the selection operation control can provide options of the preset time duration in the form of a drop-down list, and the user can select a certain option. In response to the selection instruction input by the user, the time duration selected by the user is taken as the preset time duration. For another example, the selection operation control can provide options of multiple parameter-value partitions, where the user can select a parameter-value partition, and the distribution statistics display region only displays the distribution statistic result corresponding to the selected parameter-value partition. As another example, the selection operation control may be multiple options of the physiological parameters, and in response to the selection instruction input by the user, the physiological parameter selected by the user is taken as the target physiological parameter.

The distribution statistic chart/table is generated based on the parameter-value partition and the distribution statistic result. For example, the distribution statistic chart/table is the histogram with the parameter-value partition and the distribution statistic result as two coordinate axes. Alternatively, the distribution statistic chart/table may include a statistic chart such as a pie chart, or a statistic table.

FIGS. 2D and 2G shows two examples of the monitoring interface. The monitoring interface includes a state indication region 221 and the distribution statistics display region 232. The distribution statistics display region 232 displays a histogram of blood oxygen saturation. The histogram is obtained after the distribution statistics on the historical values of the blood oxygen saturation. It can be seen from the description of the histogram that the historical values are the parameter values within 24 hours (h) before the current time point.

As shown in FIG. 2D, the monitoring interface further includes the trend display region 233 on the basis of including the state indication region 231 and the distribution statistics display region 232. The trend display region 233 contains trend charts of three physiological parameters, including blood oxygen saturation (SpO2), pulse rate (PR) and perfusion index (PI). The trend chart may contain the normal parameter value range of the physiological parameter, and this range can be marked with shading and numerical values. As shown in FIG. 2D, it can be seen from the numerical scale that the normal parameter value range of the blood oxygen saturation (SpO2) includes 91%-95%, the normal parameter value range of the pulse rate (PR) includes 100 bpm to 200 bpm, and the normal parameter value range of the perfusion index (PI) is 1.0 or more.

Alternatively, a trend table of the historical values of the physiological parameter can be displayed in the trend display region. In the implementation of the trend table, the historical values of the physiological parameter may include the parameter values in a discontinuous time period. Specifically, the historical values include: the parameter values of the physiological parameter at multiple time points of different preset time durations from the current time point. It should be noted that the time points of the physiological parameter have different time durations from the current time point, but the time points of the physiological parameter may have the same time interval therebetween. In brief, the parameter values of the physiological parameter are obtained at regular intervals within a historical time period.

FIG. 2G shows yet another example of the monitoring interface. The monitoring interface is different from FIG. 2D in that the trend display region displays the trend tables of the blood oxygen saturation (SpO2), the pulse rate (PR) and the perfusion index (PI). The trend tables show the parameter values of the blood oxygen saturation (SpO2), the pulse rate (PR) and the perfusion index (PI) at multiple different historical time points.

This disclosure further provides a monitoring device, including:
a displayer configured to display information; and
a processor, which executes a program instruction to implement the steps of the method for displaying monitoring information in the forgoing embodiments.

The monitoring device mentioned in this disclosure is not limited to monitors, but can also be invasive/non-invasive ventilators, anesthesia machines, defibrillators, nurse stations, central stations, and other devices with a monitoring function. Of course, it can also be the installation and operation of clinical data monitoring and analysis software, or a computer terminal or a mobile terminal that implements the method provided in the above embodiment. The following embodiment mainly takes a monitor as an example for description.

A specific example of the monitor is shown in FIG. 4. FIG. 4 provides a system framework diagram of a parameter processing module in a multi-parameter monitor.

The multi-parameter monitor has an independent housing. A housing panel has a sensor interface zone in which multiple sensor interfaces are integrated for connecting with external physiological parameter sensor accessories 411. The housing panel further includes a small IXD display zone, a displayer 418, an input interface circuit 420, an alarm circuit 419 (such as an LED alarm zone), etc. The parameter processing module is used as an external communication and power source interface for communicating with a main unit and taking power from the main unit. The parameter processing module also supports a build-out parameter module, can form a plug-in monitor main unit by means of inserting the parameter module, can be used as part of the monitor, or can be connected to the main unit via a cable, with the build-out parameter module being used as an external accessory of the monitor. In addition, the multi-parameter monitor comprises a memory 417 for storing computer programs and various data generated during the related monitoring process.

The internal circuit of the parameter processing module is disposed in the housing, as shown in FIG. 4, and includes signal acquisition circuits 412 corresponding to at least two physiological parameters, a front-end signal processing circuit 413, and a main processor 415.

The main processor 415 can implement the steps related to processing in each method for displaying monitoring information described above.

The signal acquisition circuits 412 may be selected from an electrocardiogram circuit, a respiration circuit, a body temperature circuit, a blood oxygen saturation circuit, a non-invasive blood pressure circuit, an invasive blood pressure circuit, etc. The signal acquisition circuits 412 are respectively electrically connected to the corresponding sensor interfaces and are used to be electrically connected to the sensor accessories 411 corresponding to different physiological parameters, with an output end thereof being coupled to the front-end signal processor. A communication port of the front-end signal processor is coupled to the main processor, and the main processor is electrically connected to the external communication and power source interface.

Various physiological parameter measurement circuits can use common circuits in the prior art. The front-end signal processor completes sampling and analog-to-digital conversion of the output signal of the signal acquisition circuit, and outputs control signals to control the measurement process of the physiological signals. These parameters include but are not limited to parameters of electrocardiogram, respiration, body temperature, blood oxygen saturation, non-invasive blood pressure, and invasive blood pressure.

The front-end signal processor can be realized by a single-chip microcomputer or other semiconductor devices, and can also be realized by ASIC or FPGA. The front-end signal processor can be powered by an isolated power source. The sampled data is simply processed and packaged, and then sent to the main processor through the isolated communication interface. For example, the front-end signal processor circuit can be coupled to the main processor 415 through the isolated power source and communication interface 414.

The reason that the front-end signal processor is powered by an isolated power source is that the DC/DC power source isolated by a transformer plays a role in isolating the patient from the power supply device, and the main purpose is: 1. isolating the patient, in which the application part is floated above the ground through the isolation transformer such that the leakage current of the patient is small enough; and 2. preventing the voltage or energy in the application of defibrillation or electric scalpel from affecting the boards and devices of the intermediate circuit such as the main control board (guaranteed by creepage distance and electrical clearance).

The main processor completes the calculation of physiological parameters, and sends the calculation results and waveforms of the parameters to the main unit (such as a main unit with a displayer, a PC, a central station, etc.) through the external communication and power source interface. The external communication and power source interface 416 may be one or a combination of local area network interfaces composed of Ethernet, token ring, token bus, and an optical fiber distributed data interface (FDDI) as the backbone of these three networks, may also be one or a combination of wireless interfaces such as infrared, Bluetooth, WIFI, and WMTS communication, or may also be one or a combination of wired data connection interfaces such as RS232 and USB.

The external communication and power source interface 416 may also be one or a combination of the wireless data transmission interface and the wired data transmission interface. The main unit may be any computer device such as the main unit of the monitor, an electrocardiogram ultrasonic diagnosis instrument, a computer, etc., and a monitoring device can be formed by means of installing with matching software. The main unit may also be a communication device, such as a mobile phone, and the parameter processing module sends data to a mobile phone that supports Bluetooth communication via a Bluetooth interface so as to realize remote data transmission.

In addition, this disclosure provides a readable storage medium with a computer program stored thereon, and the method for displaying monitoring information above is implemented when the computer program is executed by a processor.

The description has been made with reference to various exemplary embodiments herein. However, those skilled in the art would have appreciated that changes and modifications could have been made to the exemplary embodiments without departing from the scope herein. For example, various operation steps and components for performing operation steps may be implemented in different ways according to a specific application or considering any number of cost functions associated with the operation of the system (for example, one or more steps may be deleted, modified or incorporated into other steps).

The terms “first”, “second”, etc. in the specification and the claims herein as well as the above accompanying drawings are used to distinguish different objects, rather than to describe a specific order. In addition, the terms “comprising”, “having”, and any variations thereof are intended to cover non-exclusive inclusion. For example, a process, a method, a system, a product, or a device that includes a series of steps or units is not limited to the listed steps or units, but optionally further includes unlisted steps or units, or optionally further includes other steps or units inherent in these processes, methods, or devices.

In addition, as understood by those skilled in the art, the principles herein may be reflected in a computer program product on a computer-readable storage medium that is pre-installed with computer-readable program codes. Any tangible, non-transitory computer-readable storage medium can be used, including magnetic storage devices (hard disks, floppy disks, etc.), optical storage devices (CD-ROM, DVD, Blu Ray disks, etc.), flash memory, and/or the like. These computer program instructions can be loaded onto a general-purpose computer, a dedicated computer, or other programmable data processing device to form a machine, so that these instructions executed on a computer or other programmable data processing apparatus can generate an apparatus that implements a specified function. These computer program instructions can also be stored in a computer-readable memory that can instruct a computer or other programmable data processing device to operate in a specific manner, so that the instructions stored in the computer-readable memory can form a manufactured product, including an implementation apparatus that implements a specified function. The computer program instructions can also be loaded onto a computer or other programmable data processing device, so that a series of operating steps are performed on the computer or other programmable device to produce a computer-implemented process, so that the instructions executed on a computer or other programmable data processing apparatus can provide steps for implementing specified functions.

The foregoing specific description has been described with reference to various embodiments. However, those skilled in the art would have appreciated that various modifications and changes could have been made without departing from the scope of the present disclosure. Therefore, consideration of the present disclosure will be in an illustrative rather than a restrictive sense, and all such modifications will be included within the scope thereof. Likewise, the advantages of various embodiments, other advantages, and the solutions to problems have been described above. However, the benefits, advantages, solutions to problems, and any elements that can produce these, or solutions that make them more explicit, should not be interpreted as critical, necessary, or essential. The term “comprising” and any other variants thereof used herein are non-exclusive, so that the process, method, document, or device that includes a list of elements includes not only these elements, but also other elements that are not explicitly listed or do not belong to the process, method, system, document, or device. Furthermore, the term “coupling” and any other variations thereof used herein refer to physical connection, electrical connection, magnetic connection, optical connection, communication connection, functional connection, and/or any other connection.

The above-mentioned examples merely represent several embodiments, giving specifics and details thereof, but should not be understood as limiting the scope of the present disclosure thereby. It should be noted that those of ordinary skill in the art would have also made several variations and improvements without departing from the concept of the present disclosure, and these variations and improvements would all fall within the scope of protection of the present disclosure. Therefore, the scope of protection of the present disclosure shall be in accordance with the appended claims.

What is claimed is:

1. A method for displaying monitoring information on a monitoring device, comprising:
acquiring, by at least one sensor, measurement data of at least one physiological parameter of a monitored object;
obtaining, by a main processor, data of the at least one physiological parameter within a pre-set time duration from the measurement data of the at least one physiological parameter;
performing, by the main processor, distribution statistics on the data of the at least one physiological parameter within the pre-set time duration according to a plurality of parameter-value partitions, and determining a distribution statistic result corresponding to the plurality of parameter-value partitions, comprises:
determining a plurality of numerical intervals of the at least one physiological parameter as the plurality of parameter-value partitions; wherein the parameter-value partition comprises a treatment target partition corresponding to a first numerical interval of a treatment target value of the at least one physiological parameter; and
determining a respective ratio of a number of the data of the at least one physiological parameter within the pre-set time duration falling into each of the plurality of numerical intervals corresponding to the parameter-value partitions, thereby obtaining a plurality of respective ratios for the plurality of numerical intervals;
wherein the distribution statistic result is a histogram having a plurality of bars, each of the plurality of bars respectively corresponding to one of the plurality of numerical intervals and one of the plurality of respective ratios; one single bar among the plurality of bars corresponds to the treatment target partition and a first ratio among the plurality of ratios, and said one single bar has a height determined by the first ratio of the treatment target partition;
providing, by the main processor, a monitoring interface that comprises a measurement data display region and a distribution statistics display region at the monitoring interface;
displaying the measurement data of the at least one physiological parameter in the measurement data display region of the monitoring interface; and
displaying the histogram in the distribution statistics display region of the monitoring interface, wherein the plurality of ratios are displayed as respective percentage values adjacent to the plurality of bars and the plurality of numerical intervals are displayed as one coordinate axe of the histogram.

2. The method of claim 1, further comprising:
providing a selection operation control, which provides options of the plurality of parameter-value partitions; and
determining, according to the selected parameter-value partition, one or more of the plurality bars of the histogram to be displayed in the distribution statistics display region.

3. The method of claim 1, further comprising:
providing a selection operation control, which provides options of the pre-set time duration, in the distribution statistics display region; and
in response to a selection instruction input by a user through the options of the pre-set time duration, determining a value of the pre-set time duration.

4. The method of claim 1, wherein the parameter-value partition further comprises a non-treatment-target partition corresponding to a second numerical interval, the first numerical interval corresponding to the treatment target partition and the second numerical interval corresponding to the non-treatment-target partition are different in widths.

5. The method of claim 4, further comprises: displaying the non-treatment-target partition with a first display pattern, and displaying the treatment target partition with a second display pattern that is different from the first display pattern.

6. The method of claim 1, wherein the monitoring device comprises a front-end signal processor powered by an isolated power source and coupled to the main processor via a communication interface, the main processor powered by a power supply device different from the isolated power source;
acquiring, by at least one sensor, measurement data of at least one physiological parameter of a monitored object comprises: collecting, by the front-end signal processor via the at least one sensor, measurement data of the at least one physiological parameters parameter of the monitored object, wherein the front-end signal processor is powered by the isolated power source that isolates the monitored object from the power supply device of the main processor.

7. The method of claim 1, wherein
the monitoring interface further comprises a state indication region that displays at least one graphical state indicator with multiple indication blocks, each of the multiple indication blocks respectively corresponds to a parameter value ranges of the at least one physiological parameter, and the multiple indication blocks are displayed in the state indication region in an orderly arrangement according to a numerical value of the parameter value ranges, of the at least one physiological parameter, corresponding to the indication blocks; and
the method further comprises determining a parameter value range to which the measurement data of the at least one physiological parameter belongs; and indicating an indication block of the graphical state indicator corresponding to the parameter value range to which the measurement data of the at least one physiological parameter belongs.

8. The method of claim 7, wherein
the multiple indication blocks comprise a treatment target indication block which represents an expected state of the monitored object with respect to the at least one physiological parameter; and
indicating an indication block of the graphical state indicator corresponding to the parameter value range to which the measurement data of the at least one physiological parameter belongs comprises:
when the parameter value range to which the measurement data of the at least one physiological parameter belongs is a parameter value range corresponding to the treatment target indication block, displaying the treatment target indication block in a display and output mode different from other indication blocks.

9. The method of claim 8, further comprising:
respectively highlighting, by the main processor, the treatment target partition in the distribution statistics display region and the treatment target indication block in the state indication region to correlate the treatment target partition with the treatment target indication block.

10. The method of claim 7, wherein indicating an indication block of the graphical state indicator corresponding to the parameter value range to which the measurement data of the at least one physiological parameter belongs comprises:
displaying an indication icon at a position of the indication block corresponding to the parameter value range to which the measurement data of the at least one physiological parameter belongs.

11. The method of claim 7, wherein
the multiple indication blocks comprise a treatment target indication block corresponding to a parameter value range of a treatment target value of the at least one physiological parameter; and
the method further comprises: when the measurement data of the at least one physiological parameter is determined to change from a parameter value range to which a non-treatment-target value belongs to a parameter value range to which the treatment target value belongs, displaying the treatment target indication block in a preset display pattern different from an original display pattern.

12. The method of claim 7, further comprising:
displaying a parameter pointer in the state indication region, with a first end of the parameter pointer indicating a position of an indication block corresponding to a parameter value range to which the measurement data of the at least one physiological parameter belongs.

13. The method of claim 12, wherein:
the multiple indication blocks of the graphical state indicator are arranged in a circular arc shape, and a second end of the parameter pointer is displayed at a center position corresponding to the circular arc.

14. The method of claim 7, wherein the numerical interval corresponding to each bar of the histogram is identical in range relative to the parameter value range of each indication block.

15. The method of claim 7, wherein the number of the plurality of the bars is equal to the number of the multiple indication blocks.

16. The method of claim 1, further comprising:
generating at least one of a corresponding waveform chart or a trend chart according to the data of the at least one physiological parameter within the pre-set time duration; and
displaying the at least one of the corresponding waveform chart or the trend chart in the measurement data display region; or, generating a trend display region at the monitoring interface, and displaying at least one of the trend chart or a trend table of the at least one physiological parameter in the trend display region.

17. The method of claim 16, further comprising:
switching between the trend chart and the trend table displayed in the trend display region in response to a sliding operation input by a user.

18. The method of claim 16, further comprising:
refreshing the at least one of the trend chart or the trend table displayed in the trend display region in response to a pre-set trigger condition; or
synchronously refreshing the distribution statistic result, at least one of the trend chart or the trend table after the measurement data of the at least one physiological parameter is updated.

19. The method of claim 1, wherein the respective percentage values are displayed above the plurality of bars and the plurality of numerical intervals are displayed below the plurality of bars in the distribution statistics display region.

20. The method of claim 1, wherein determining a respective ratio of a number of the data of the at least one physiological parameter within the pre-set time duration falling into each of the plurality of numerical intervals corresponding to the parameter-value partition:
determining, for each of the plurality of numerical intervals, a respective ratio of (i) a number of data points of the at least one physiological parameter for the respective numerical interval and (ii) a total number of data points of the at least one physiological parameter within the pre-set time duration as the distribution statistic result, thereby obtaining the plurality of respective ratios for the plurality of numerical intervals.
